(19) United States
(12) Patent Application Publication
(10) Pub. No.: US 2022/0298696 A1
(43) Pub. Date: Sep. 22, 2022

(54) METHOD FOR EVALUATING FIBER PRODUCTS

(71) Applicant: Toray Industries, Inc., Tokyo (JP)

(72) Inventors: Chika Kimura, Shiga (JP); Keigo Toyabe, Shiga (JP); Saori Igakura, Shiga (JP); Hidetoshi Suzuki, Shiga (JP)

(21) Appl. No.: 17/636,565

(22) PCT Filed: Aug. 19, 2020
(86) PCT No.: PCT/JP2020/031220

§ 371 Date: Feb. 18, 2022

(30) Foreign Application Priority Data
Aug. 30, 2019 (JP) 2019-158068

(51) Int. Cl. D06F 34/20; D06F 39/10

(52) U.S. Cl. CPC D06F 34/20; D06F 39/10; D06F 2103/42

(57) ABSTRACT

A method evaluates a fiber product and includes washing at least one fiber product by a washing machine and washing conditions defined in ISO 6330 (2012), collecting fiber fragments discharged from a wastewater outlet of the washing machine by a filter with a sieve opening of 5 to 20 μm, and measuring a weight of the fiber fragments.

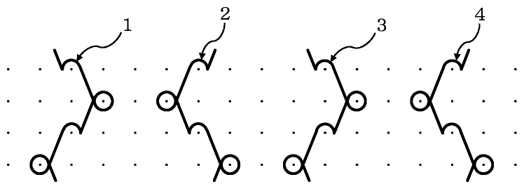

### TECHNICAL FIELD

This disclosure relates to a method of evaluating a fiber product.

### BACKGROUND

Recently, there is a concern about an adverse effect on the ecosystem due to plastic waste in oceans or rivers being taken into organisms. In particular, there is a concern that a plastic container is microminiaturized by ultraviolet rays or the like to become micro-sized plastic fragments. Waste reduction and relevance to micro-plastic problems are also discussed for all plastic products.

Synthetic fibers are often used in a fiber product currently on the market, in particular, a functional fiber product manufactured for sports and outdoor applications. The synthetic fibers may fall out as laundry fragments from a cut portion or the like during washing. For example, a bulky mid-layer having a heat-retaining property represented by a fleece is known. This is generally produced by subjecting a surface of a fabric to a raising process and fluffing fibers. This material may cause fiber fragments to fall out from a raised portion as well as the cut portion during washing, and tends to generate more fiber fragments than a non-raised material.

JIS L1096 (2010), JIS L0844, ISO 7768 (2009) and the like have been standardized as evaluation methods for washing. All such methods are test methods focusing on the quality of an object to be washed such as dimensional stability, washing fastness, wrinkle characteristics or the like of the object to be washed. In addition, since a fiber product is exposed to high mechanical stress such as washing to cause damage and the like, a standard test for detecting the mechanical stress has also been proposed (for example, Japanese Patent Laid-open Publication No. 2006-521475). On the other hand, recently, considering fiber fragments generated during washing as a problem, a washing bag having an enhanced ability to filter fiber fragments has been proposed to filter the fiber fragments (for example, Japanese Patent Laid-open Publication No. 2019-505351).

In general, the fiber fragments generated during washing or the like are removed from the washing liquid, washing wastewater or the like and then discarded. Considering the possibility of various problems such as an increase in waste, wastewater treatment load, and maintenance load of a washing machine, it is more preferable as the amount of fiber fragments is smaller. These problems can be solved by a fiber product that suppresses the amount of fiber fragments, but a method of evaluating a fiber product that suppresses the amount of fiber fragments generated during washing has not been proposed. In addition, although there is a difference in performance between a product having a large amount of fiber fragments and a product having a small amount of fiber fragments, there is no means for comparing these products, which is problematic.

For example, the JIS or ISO standard described above is an evaluation method focusing on an object to be washed during washing, and does not describe fiber fragments generated from the object to be washed. In addition, also in Japanese Patent Laid-open Publication No. 2006-521475, an evaluation target is a device, and there is no description of a method of evaluating a fiber product or fiber fragments. On the other hand, Japanese Patent Laid-open Publication No. 2019-505351 is focused on fiber fragments and describes that a fiber product is put in a washing bag and washed, and fiber fragments generated from the fiber product are not flown into wastewater. However, there is no description of a method of evaluating fiber fragments generated from a fiber product. This is because it is premised on generation of fiber fragments from a fiber product, and there is no point of view to provide a method of quantitatively evaluating fiber fragments generated from a fiber product during washing and thus to create a fiber product that suppresses generation of fiber fragments.

Therefore, a new issue has arisen in creating a method of quantitatively evaluating fiber fragments generated from a fiber product with excellent reproducibility.

It could therefore be helpful to provide a method of evaluating fiber fragments generated from a fiber product during washing.

### SUMMARY

We Thus Provide:

(1) A method of evaluating a fiber product, including: washing at least one fiber product by a washing machine and washing conditions defined in ISO 6330 (2012); filtering fiber fragments discharged from a wastewater outlet of the washing machine by a filter with a sieve opening of 5 to 20 μm; and measuring a weight of the fiber fragments.
(2) The method of evaluating a fiber product according to (1), in which a water permeability of the filter is 300 seconds or shorter when measured in the following order, water permeability measurement procedure:

(a) a sheet cut into a circular shape having a diameter of 20 cm from the filter is folded into four and set in a triangular funnel having a diameter of 22 cm;

(b) 300 ml of distilled water is poured and a time until a last one drop falls from the funnel after an initial one drop falls from the funnel is measured; and

(c) when a next drop does not fall for longer than 5 minutes, the last one drop is determined as a last one drop.

(3) The method of evaluating a fiber product according to (1) or (2), in which a filter with a sieve opening of 150 to 5,000 μm is further provided between the wastewater outlet of the washing machine and the filter.

(4) The method of evaluating a fiber product according to any one of (1) to (3), in which the washing machine is cleaned by performing each of rinse and drain at least once before the washing.

(5) The method of evaluating a fiber product according to any one of (1) to (4), in which a detergent is not put in the washing.

(6) The method of evaluating a fiber product according to any one of (1) to (5), in which one fiber product is used in the washing.

(7) The method of evaluating a fiber product according to any one of (1) to (6), in which the washing machine is a C-type standard washing machine.

(8) The method of evaluating a fiber product according to any one of (1) to (7), in which the washing condition is a 4N method.

Our method evaluates a fiber product to quantitatively evaluate the amount of fiber fragments generated with excellent reproducibility. Therefore, a fiber product that suppresses fiber fragments is easily created.

### BRIEF DESCRIPTION OF THE DRAWING

The FIGURE is a view of a knitted pattern of each of a non-elastic fiber and an elastic fiber in Production Example 1.

### DESCRIPTION OF REFERENCE SIGNS

1: First non-elastic fiber

2: Second non-elastic fiber

3: First elastic fiber

4: Second elastic fiber

### DETAILED DESCRIPTION

A fiber product to be evaluated is not particularly limited, and it is preferable that at least a part of a fabric included in the fiber product contains synthetic fibers. The fabric may be a woven fabric, a knitted fabric, or a non-woven fabric. When the fabric is a woven fabric, a weave pattern is not particularly limited, and examples thereof include plain weave, twill weave, satin weave, modified plain weave, modified twill weave, modified satin weave, fancy weave, single weave, double pattern, multiple pattern, warp pile weave, weft pile weave, and interlaced weave. In addition, the fabric is a knitted fabric, a knitted pattern is not particularly limited, and examples thereof include circular knitted fabric, weft knitted fabric, warp knitted fabric (including tricot fabric and russell fabric), pile fabric, plain stitch, plain knit, rib-knit, smooth knit (double knit), rib stitch, purl knit, denbigh pattern, cord pattern, atlas pattern, chain pattern, and insertion pattern.

Examples of the synthetic fiber constituting the fabric used for the fiber product include a filament yarn and a spun yarn. The spun yarn may contain natural fibers as long as it at least partially contains various synthetic fibers. The filament yarn includes a drawn yarn and various twisted yarns. The type of twisted yarn is not particularly limited, and examples thereof include a false twisted yarn, a false twisted-fused yarn, and a medium twisted yarn.

A material of the synthetic fiber is not particularly limited. Examples thereof include polymers such as polyethylene terephthalate, polyethylene naphthalate, polybutylene terephthalate, polytrimethylene terephthalate, polypropylene, polyolefin, polycarbonate, polyacrylate, polyamide, polylactic acid, polyurethane, and polyphenylene sulfide, and copolymers thereof. These materials may contain various additives such as an inorganic substance such as titanium oxide, silica, or barium oxide, a colorant such as carbon black, a dye, or a pigment, a flame retardant, a fluorescent brightener, an antioxidant, and an ultraviolet absorber.

A cross-sectional shape of the fiber is not particularly limited, and may be various cross-sectional shapes such as a flat cross section, a polygonal cross section such as a triangle, a quadrangle, a hexagon, or an octagon, a dumbbell-shaped cross section partially having concave and convex portions, a Y-shaped cross section, and a star-shaped cross section, in addition to a perfect circular cross section.

A method of dyeing the fabric is not particularly limited. Examples thereof include scouring, relaxation, heat setting, dyeing processing, weight reduction processing, and functional processing. In the functional processing, water repellency, antistatic processing, flame retardancy, moisture absorption, antistatic processing, antibacterial processing, flexible finishing, and other known functional processing can be performed, if necessary. When the synthetic fiber contained in the fabric includes polyurethane fibers, heat setting is preferably performed at 190° C. or higher. When the temperature is equal to or higher than this temperature, the polyurethane fibers are fused, and it is possible to suppress fraying of the fibers and generation of the fiber fragments that fall off.

A method of cutting the fabric is not particularly limited, and it is preferable to select ultrasonic cutting. In the cutting method, a cut portion is welded such that it is possible to suppress fraying of the fibers and generation of the fiber fragments that fall off.

A method of sewing the fiber product is not particularly limited, and a piping treatment or an embedding treatment using a sealing tape is preferable. By applying these treatments, it is possible to prevent the cut portion of the fabric from being exposed during washing and to suppress fraying of the fibers and generation of the fiber fragments that fall off.

In addition, the fiber product after sewing is preferably subjected to a post-treatment of any of washing, air blowing, and air suction. When these treatments are performed, the fiber fragments attached to the cut portion of the fabric and the fabric surface can fall off in advance, and the amount of fiber fragments generated during washing can be suppressed.

One aspect of a method of evaluating a fiber product is a method of performing a washing test of a fiber product, filtering fiber fragments using a filter attached to a drain hose or the like of a washing machine, for example, a filtering bag (filter), and evaluating the weight of the fiber fragments. Evaluation accuracy can be improved by evaluating the weight of the generated fiber fragments instead of evaluating a change in weight of the fiber product that is an object to be washed. A specific method is as follows.

It is preferable that before the evaluation, washing is performed according to ISO 6330 (2012) without putting an object to be washed or a detergent in the washing machine and the washing machine is cleaned. It is possible to prevent the influence of the residue of the fiber fragments and the like due to the washing performed in advance and improve the reproducibility of the evaluation result. Conventional JIS or ISO to evaluate an object to be washed does not require the method described above, and thus, the above description is not defined therein. However, it is a preferable characteristic step in our evaluation method of evaluating fiber fragments. It is more preferable to clean the washing machine by performing rinsing and dehydrating steps once or more each without putting an object to be washed and a detergent. In addition, it is preferable that conditions are the same as the washing conditions for evaluation or the amount of water and washing machine power are set to the maximum.

In our evaluation method, a washing machine defined in ISO 6330 (2012) is used. There are various different types and shapes of washing machines, and the washing machines have different physical actions (washing machine power) applied to an object to be washed during washing. Among various washing machines used in general households, the washing machine defined in ISO 6330 (2012) has a basic function and thus is used for evaluation of a fiber product. An A-type standard washing machine, a B-type standard washing machine, and a C-type standard washing machine are defined in ISO 6330 (2012), and any one of these washing machines can be used. Among them, an A-type standard washing machine or a C-type standard washing machine that is actually used at home in many countries can be preferably used in the evaluation method. Further, it is possible to more preferably use a C-type standard washing machine capable of more accurately measuring the fiber fragments by suppressing re-attachment of the fallen fiber fragments to the object to be washed or the like due to a large amount of water used during washing.

The washing is performed under conditions defined in ISO 6330 (2012). Several types of washing conditions are defined for each of the washing machines described above, and any of the washing conditions can be adopted. For example, 13 types of washing methods and 7 types of washing methods are defined for an A-type standard washing machine and a C-type standard washing machine that are preferably used, respectively. Among them, as the washing conditions in the present invention, a 4N method, 4M method, 3N method, or 3M method for the A-type standard washing machine and a 4N method, 4M method, 3N method, or 3M method for the C-type standard washing machine are preferable, and the 4N method for the C-type standard washing machine is more preferable, in terms of high reproducibility of the amount of fiber fragments generated from a fiber product and suitability for evaluating the amount of fiber fragments. In general, when the washing machine power is too weak due to a washing machine or washing conditions, a proportion of a relatively large incidentally attached yarn or the like in the filtered fiber fragments increases, and accurate evaluation tends to be difficult. On the contrary, when the washing machine power is too strong, fragments other than the fiber fragments generated by washing are generated due to damage of the fiber product, and accurate evaluation also tends to be difficult. By the washing machine and washing conditions, in particular, the preferred washing machine and washing conditions described above, potential fiber fragments that may be generated during washing of a fiber product are efficiently generated, and it is easy to filter and evaluate the fiber fragments by a filter with a sieve opening defined in the present invention.

The fiber fragments discharged from a wastewater outlet of the washing machine are filtered by attaching a filter (a “first filter”) with a sieve opening of 5 to 20 μm to a drain hose or the like of the washing machine. According to the washing machine and washing conditions adopted in the evaluation method, the weight of the fiber fragments can be evaluated with excellent reproducibility by setting the sieve opening within our range. When the sieve opening is less than 5 μm, a drainage speed is lowered and the fiber fragments are likely to remain in the washing machine, and it is difficult to collect and evaluate the generated fiber fragments with excellent reproducibility. In addition, it is required to set the sieve opening to 20 μm or less to filter the fiber fragments. From the viewpoint of a balance, the sieve opening is preferably set to 9 μm or more. In addition, the sieve opening is preferably set to 15 μm or less, and is more preferably set to 12 μm or less.

It is preferable that in addition to the first filter, a filter with a sieve opening of 150 to 5,000 μm (a “second filter”) is further provided between the wastewater outlet of the washing machine and the first filter. The fiber fragments filtered by the first filter can be used to evaluate fine fiber fragments that fall off from the fiber product. However, when large fiber fragments that are not preferable as an evaluation target are generated, the large fiber fragments affect the evaluation result. On the other hand, the evaluation using the first filter becomes preferably quantitative with excellent reproducibility by removing the influence of large fiber fragments by the second filter. It is preferable that a weight of the fiber fragments filtered by the second filter is similarly measured as reference information and the measured weight is added to the evaluation result. The sieve opening can be obtained by measuring 10 points with a microscope of 500 magnifications and rounding off the average value to the first decimal place. When a hole of the sieve opening has a round shape, a maximum diameter is measured. a quadrangular shape, the longer one of a warp and a weft is measured. When a filter is incorporated into the washing machine, the incorporated filter is removed because it affects the filtering amount.

In addition, the filter is not limited to a sheet shape, a bag shape, a box shape or the like, and may have any structure as long as there is the sieve opening as described above and water permeates. Specific examples thereof include a metal mesh, a perforated metal sheet, a perforated filter, a non-woven fabric, a woven fabric, and a knitted fabric, and a woven fabric is more preferably used because it is excellent in versatility, water permeability, and filtering performance and has high reproducibility.

A material of the filter may be either a metal or plastic.

The plastic is not particularly limited. Examples thereof include polymers such as polyethylene terephthalate, polyethylene naphthalate, polybutylene terephthalate, polytrimethylene terephthalate, polypropylene, polyolefin, polycarbonate, polyacrylate, polyamide, polylactic acid, polyurethane, and polyphenylene sulfide, and copolymers thereof.

The water permeability of the filter is preferably 300 seconds or shorter. When the water permeability is 300 seconds or shorter, a decrease in drainage speed from the washing machine is suppressed, and an amount of fiber fragments remaining in the washing machine is small so that the fiber fragments can be collected with excellent reproducibility. The water permeability is more preferably 200 seconds or shorter, and still more preferably 100 seconds or shorter. In addition, a lower limit thereof is not particularly limited, and is substantially 5 seconds or longer. The water permeability can be appropriately adjusted by a porosity, water absorbency and the like of the filter. When the filter has a fiber structure, the water permeability can be adjusted by also a density, fineness, and pattern.

The water permeability is measured by the following evaluation method.

(a) A sheet cut into a circular shape having a diameter of 20 cm from the filter is folded into four and set in a triangular funnel having a diameter of 22 cm. That is, the sheet is folded in half twice, and one to be into a bag shape is opened in a conical shape and set in a triangular funnel. This is a method similar to a four-fold method of a filter paper although there are different portions such as a folding portion and a torn portion.

(b) 300 ml of distilled water is poured and a time until a last one drop falls from the funnel after an initial one drop falls from the funnel is measured.

(c) When a next drop does not fall for longer than 5 minutes, the last one drop is determined as a last one drop.

Such a filter is not special, and may be appropriately selected from commercially available sheets and the like. A specific example thereof includes a filtering bag produced using a “nylon screen” NY10-HC (manufactured by FLON INDUSTRY).

At least one fiber product to be evaluated is put in a washing machine in a state where a filter is attached, and the fiber product is washed by the washing machine and washing conditions described above. However, a detergent and a loading fabric are not used. After the washing, a weight of fiber fragments attached to a filter, for example, the “nylon screen” is measured. One fiber product refers to one fiber product regardless of a shape, size, and weight. The amount of fiber fragments generated does not necessarily depend on the size and weight, and definition of the number of fiber products is more excellent in reproducibility or quantitativeness. In principle, it is preferable to perform relative comparison in the same or similar product group. In addition, it is more preferable to append a weight, shape, and size of the fiber product, and a fiber fragment ratio obtained by the amount of fiber fragments in the weight of the fiber product to the evaluation result in compared to another fiber product. In addition, to improve reproducibility or quantitativeness, it is preferable to prepare a fiber product to be a reference and similarly wash and compare the fiber product. In addition, the number of fiber products is at least one, and it is also allowable to wash a plurality of fiber products at the same time. On the other hand, to prepare 2 kg of a fiber product as described in ISO 6330 (2012), in a case of 100 g of one piece of inner wear, it is required to prepare 20 pieces. In consideration of this, the number of fiber products is preferably one from the viewpoint of convenience of evaluation.

A method of measuring the weight of the fiber fragments collected by the filter is not particularly limited to a method in which a filter is dried to measure a weight, a method in which fiber fragments are washed off from a filter using pure water and then the fiber fragments are collected through filtration by a filter with a sieve opening of 0.1 to 20 μm and the like. A method of measuring a weight of fiber fragments by drying a filter is preferable because it is simple and hardly causes an operation error. In addition, a method of collecting fiber fragments by filtration is preferable because there is no occurrence of an error caused by a change in weight of the fiber fragments due to moisture absorption and desorption of the filter, outflow of a material of the filter and the like. To collect the fibers filtered by the filter, the sieve opening of the filter may be set to be smaller than the sieve opening of the filter. A specific example of the method of collecting fiber fragments by filtration is described below. After absolute drying, suction filtration is performed using a filter whose weight is measured in advance, for example, a polycarbonate membrane (K040A047A, manufactured by Advantec Toyo Kaisha, Ltd.). The filter and the fiber fragments after filtration are dried at 105° C. for 1 hour, the weight is measured, and a difference from the weight before filtration is taken as the amount of fiber fragments. As for conditions of the absolute drying and weight measurement, heating is performed at 105° C. for 1 hour, and then, the weight is measured after the temperature and humidity are controlled to 20° C. and 65% RH, respectively. The amount of fiber fragments filtered in the present evaluation is preferably 10.0 (mg/one fiber product) or less, and more preferably 6.0 (mg/one fiber product) or less as a target fiber product.

A method in which one fiber product is put in a washing bag and washed, and fiber fragments attached to the washing bag are filtered and evaluated (a “washing bag method”) is also considered. Compared to our evaluation method, the method of measuring the amount of fiber fragments in the filtering method is as follows. A “nylon screen” NY10-HC (manufactured by FLON INDUSTRY) is used to prepare an isosceles triangular washing bag having one side of 1 m. A fiber product to be evaluated is put in the washing bag, and washing is performed according to an ISO 6330C 4N method. However, a detergent and a loading fabric are not used. After the washing, the fiber product is removed, and suction filtration is performed using a polycarbonate membrane (K040A047A, manufactured by Advantec Toyo Kaisha, Ltd.) in which a weight of the fiber fragments attached to the washing bag is measured in advance. The polycarbonate membrane and the fiber fragments after filtration are dried at 105° C. for 1 hour, the weight is measured, and a difference from the weight before filtration is taken as the amount of fiber fragments.

Even in this washing bag method, the fiber fragments can be evaluated. However, when the fiber product is put in the washing bag, washing machine power is weakened, and an amount of fiber fragments generated is smaller than that in actual washing. Further, since the fiber fragments are re-attached to the fiber product, reproducibility of the amount of fiber fragments is poor, and it is difficult to relatively compare the amount of fiber fragments generated from the fiber product.

Since the reproducibility is deteriorated due to the influence of a detergent on the amount of fiber fragments generated, washing is preferably performed in an amount equal to or less than the amount defined in JIS or ISO, and washing is more preferably performed without putting a detergent. When a detergent is put, it is required to consider insoluble components of the detergent. In addition, since fiber fragments are also generated from a loading fabric, it is preferable not to use the loading fabric.

### EXAMPLES

Hereinafter, our method of evaluating a fiber product will be specifically described with reference to Examples.

### (1) Water Permeability

(a) A sheet cut into a circular shape having a diameter of 20 cm from a filter was folded into four and set in a triangular funnel having a diameter of 22 cm.

(b) 300 ml of distilled water was poured and a time until a last one drop fell from the funnel after an initial one drop fell from the funnel was measured.

(c) When a next drop did not fall for longer than 5 minutes, the last one drop was determined as a last one drop.

### (2) Sieve Opening

A surface of the filter was imaged at a magnification of 500 times using a digital microscope VHX-7000 (manufactured by Keyence Corporation), and 10 opening portions were measured. An average value of the obtained values was rounded off to the first decimal place and taken as a value of a sieve opening. When the hole had a round shape, a maximum diameter was measured, and when the hole had a square shape, the longer diameter was measured.

### (3) Measurement of Weight of Fiber Fragments

Suction filtration was performed on an aqueous solution containing fiber fragments using a polycarbonate membrane (K040A047A, hole diameter of 0.4 μm, manufactured by Advantec Toyo Kaisha, Ltd.) whose weight was measured in advance. The polycarbonate membrane and the fiber fragments after filtration were dried at 105° C. for 1 hour, the weight was measured, and a difference from the weight before filtration was taken as the amount of fiber fragments.

### Production Example 1 of Fiber Product

A warp knitted fabric composed of knitted loops formed of non-elastic fibers obtained using nylon fibers (40 dtex-34F) and elastic fibers obtained using urethane fibers (22 dtex) was produced. The warp knitted fabric is knitted with a knitted pattern illustrated in the FIGURE. That is, the warp knitted fabric has a non-elastic fiber knitted pattern formed of a pair of half-set knits of first non-elastic fibers 1 and second non-elastic fibers 2 threaded in one-in and one-out half sets through a first reed and a second reed, respectively, and an elastic fiber knitted pattern formed of a pair of half-set knits of first elastic fibers 3 and second elastic fibers 4 threaded in one-in and one-out half sets through a third reed and a fourth reed, respectively. In addition, as illustrated in the FIGURE, in the non-elastic fiber knitted pattern, the first non-elastic fiber 1 is composed of repeating units of 10/12/23/21//, and the second non-elastic fiber 2 is composed of repeating units of 23/21/10/12//. In the non-elastic fiber knitted pattern, four courses are repeated units, and each repeating unit is deflected left and right within a range of two wales. As illustrated in the FIGURE, the elastic fiber knitted pattern formed of the first elastic fibers 3 and the second elastic fibers 4 is also the same knitted pattern as the non-elastic fiber knitted pattern. As for the mixing ratio of the materials, nylon is 84% and polyurethane is 16%. The obtained fabric was subjected to a heat treatment at 190° C. for 1 minute. Another processing was performed according to dyeing processing conditions of a common warp knitted fabric to obtain a knitted fabric. A common T-shirt having a V-shaped collar was produced using the obtained knitted fabric. In the production of the T-shirt, a collar, a hem, and a cuff were not sewed, and only common cutting was finished. The sewing of an armpit portion, a front and back body joint portion, and a cuff and body joint portion was performed by a common treatment method for the T-shirt.

### Production Example 2 of Fiber Product

A single circular knitting machine was used to produce a circular knitted fabric with a plain knitted pattern using polyester fibers (66 dtex-96F) as non-elastic fibers and polyurethane fibers (22 dtex) as elastic fibers. As for a mixing ratio of the materials, polyester is 90% and polyurethane is 10%. Processing was performed on the obtained fabric according to dyeing processing conditions of a common circular knitted fabric to obtain a knitted fabric. A common T-shirt having a U-shaped collar was produced using the obtained knitted fabric. In the production of the T-shirt, unlike Production Example 1, all a collar, a hem, and a cuff, an armpit portion, a front and back body joint portion, and a cuff and a body joint portion were sewed by a common method for a T-shirt.

### Production Example 3 of Fiber Product

A 1:1 interknitted fabric with a plain knitted pattern was produced using acryl/rayon spun yarns (metric number: 1/64) for knitted loops 1 formed of non-elastic fibers, and using polyester fibers (84 dtex-72F) and polyurethane elastic fibers (33 dtex) for knitted loops 2 formed of non-elastic fibers and elastic fibers. As for a mixing ratio of the materials, acryl is 28%, rayon is 34%, polyester is 33%, and polyurethane is 5%. Processing was performed on the obtained fabric according to dyeing processing conditions of a common circular knitted fabric to obtain a knitted fabric. A common nine-tenth length T-shirt having a U-shaped collar was produced using the obtained knitted fabric, and the T-shirt was sewed in the same manner as that of Production Example 2.

### Examples 1 to 3

The fiber product obtained in each of Production Examples 1 to 3 and a C-type standard washing machine described in ISO 6330 (2012) were used. First, for cleaning of a washing machine, 7 kg of AQW-V 700E (manufactured by AQUA Co., Ltd.) was used by a C4N method of ISO 6330 (2012), and rinse and drain were performed twice without putting an object to be washed. Specifically, a course was carefully set, the amount of water was set to 40 L, a washing time was set to 15 minutes, rinse was set to two times, dehydration was set to 7 minutes, a washing water temperature was set to 40° C., and a rinsing water temperature was set to room temperature. Next, a filtering bag produced using a “nylon screen” NY10-HC (manufactured by FLON INDUSTRY) with a sieve opening of 11.3 μm and a water permeability of 82 seconds was attached to a drain hose of the washing machine. Thereafter, one fiber product to be evaluated was put in the washing machine, and washing was performed under washing conditions of a C4N method of ISO 6330. However, a detergent and a loading fabric were not used. After the washing, suction filtration of the fiber fragments attached to the “nylon screen” was performed using a polycarbonate membrane (K040A047A, manufactured by Advantec Toyo Kaisha, Ltd.) of which a weight was measured in advance. The polycarbonate membrane and the fiber fragments after filtration were dried at 105° C. for 1 hour, the weight was measured, and a difference from the weight before filtration was taken as the amount of fiber fragments generated.

The amount of fiber fragments filtered by a wastewater filtering method was 9.8 mg/one fiber product (weight of fiber fragments/weight of fiber product=114 ppm) in Example 1 using the fiber product of Production Example 1, 34.3 mg/one fiber product (279 ppm) in Example 2 using the fiber product of Production Example 2, and 72.0 mg/one fiber product (444 ppm) in Example 3 using the fiber product of Production Example 3.

In Example 1, since the polyurethane fibers were fused by the high temperature heat treatment at 190° C., we found that the generation of the fiber fragments from the cut portion was suppressed and the amount of fiber fragments generated measured after the washing was smaller than in each of Examples 2 and 3 in which the polyurethane fibers were not fused. Since the knitted fabric formed of filaments was used in Example 2 and the knitted fabric formed of staples and filaments was used in Example 3, the results were shown that the fiber fragments were likely to be generated from the staples.

### Example 4

The same operation was performed in the same manner as that of Example 1, except that the fiber product obtained by Production Example 1 was used, and a stainless steel mesh (number of meshes: 4.7, manufactured by Clever Co., Ltd.) with a sieve opening of 4,004 μm was attached between the wastewater outlet of the washing machine and the filtering bag. The obtained fiber fragments were almost equivalent to 9.6 mg/one fiber product (111 ppm), but we found that a standard deviation was small when a plurality of other fiber products obtained by Production Example 1 were evaluated.

### Comparative Examples 1 to 3

The same operation (washing bag method) was performed in the same manner as that of Example 1, except that a washing bag was used instead of attaching the filter to the drain hose. The amount of fiber fragments filtered by the washing bag method was 3.9 mg/one fiber product (45 ppm) in Comparative Example 1 using the fiber product of Production Example 1, 11.0 mg/one fiber product (89 ppm) in Comparative Example 2 using the fiber product of Production Example 2, and 10.1 mg/one fiber product (62 ppm) in Comparative Example 3 using the fiber product of Production Example 3. In Comparative Example 2, more fiber fragments were generated than in Comparative Example 3, and the results were shown that the tendency was different from those obtained in Example 2 and Example 3.

In addition, the amount of fiber fragments obtained by the washing bag method is generally smaller than that obtained by the wastewater filtering method using no washing bag, but it is because the amount of fiber fragments generated is reduced by a reduction in wafer flow applied to the fiber product by the washing bag and limitation of the flow of the fiber product during the washing test.

### Comparative Examples 4 to 6

The same operation was performed in the same manner as those of Examples 1 to 3, except that a “nylon screen” NY50-HC (manufactured by FLON INDUSTRY) with a sieve opening of 50 μm was used. The amount of fiber fragments filtered was 4.0 mg/one fiber product (45 ppm) in Comparative Example 4 using the fiber product of Production Example 1, 11.5 mg/one fiber product (93 ppm) in Comparative Example 5 using the fiber product of Production Example 2, and 21.7 mg/one fiber product in Comparative Example 6 using the fiber product of Production Example 3. It was estimated that the results were shown that the amount of fiber fragments was smaller than that in each of Examples and a small amount of the fiber fragments flowed.

### Reference Example

The same operation was performed in the same manner as that of Example 1, except that an object to be washed was not put and the cleaning of the washing machine was not performed. A calculated weight of the fiber fragments was 4.6 mg. We estimated that fibers with various colors were not mixed with the fiber fragments and the fiber product that had been washed in the past was included. The results were shown that the cleaning of the washing machine was important for evaluating the fiber fragments by washing.

1.-8. (canceled)

9. A method of evaluating a fiber product comprising:
washing at least one fiber product by a washing machine and washing conditions defined in ISO 6330 (2012);
filtering fiber fragments discharged from a wastewater outlet of the washing machine by a filter with a sieve opening of 5 to 20 μm; and
measuring a weight of the fiber fragments.

10. The method according to claim 9, wherein water permeability of the filter is 300 seconds or shorter when measured in order,
(a) a sheet cut into a circular shape having a diameter of 20 cm from the filter is folded into four and set in a triangular funnel having a diameter of 22 cm;
(b) 300 ml of distilled water is poured and a time until a last one drop falls from the funnel after an initial one drop falls from the funnel is measured; and
(c) when a next drop does not fall for longer than 5 minutes, the last one drop is determined as a last one drop.

11. The method according to claim 10, wherein a filter with a sieve opening of 150 to 5,000 μm is further provided between the wastewater outlet of the washing machine and the filter.

12. The method according to claim 9, wherein the washing machine is cleaned by performing each of rinse and drain at least once before the washing.

13. The method according to claim 9, wherein a detergent is not put in the washing.

14. The method according to claim 9, wherein one fiber product is used in the washing.

15. The method according to claim 9, wherein the washing machine is a C-type standard washing machine.

16. The method according to claim 9, wherein the washing condition is a 4N method.
